(19) United States
(12) Patent Application Publication
(10) Pub. No.: US 2007/0167856 A1
(43) Pub. Date: Jul. 19, 2007

(54) Microelectrode recording analysis and visualization for improved target localization

(75) Inventors: James McNames, Portland, OR (US); Roberto A. Santiago, Portland, OR (US); Jon Haakon Falkenberg, Portland, OR (US); Kim J. Burchiel, Portland, OR (US)

Correspondence Address: KLARQUIST SPARKMAN, LLP, 121 SW SALMON STREET, SUITE 1600, PORTLAND, OR, 97204, US

(73) Assignees: OREGON HEALTH & SCIENCE UNIVERSITY, Portland, OR (US); STATE OF OREGON ACTING BY & THROUGH THE STATE BOAR, Portland, OR (US)

(21) Appl. No.: 10/553,814

(22) PCT Filed: Apr. 19, 2004
(86) PCT No.: PCT/US04/12192

§ 371 Date: Jan. 10, 2007

Related U.S. Application Data
(60) Provisional application No. 60/464,022, filed on Apr. 18, 2003.
(60) Provisional application No. 60/533,853, filed on Dec. 31, 2003.

(51) Int. Cl. A61B 5/04

(52) U.S. Cl. 600/544

(57) ABSTRACT

Methods of processing neuronal signals include processing microelectrode recordings (MERs) or portions of MERs to provide arrays of associated values, such as estimates of power spectral density, or a marginal probability distribution, or a rate of change of a spike rate. Such arrays of values can be displayed, and a classifier can be applied to, for example, aid in associating a MER with a particular brain feature.

### CROSS REFERENCE TO RELATED APPLICATIONS

This application claims the benefit of U.S. Provisional Patent Application 60/533,853, filed Dec. 31, 2003 and U.S. Provisional Patent Application 60/464,022, filed Apr. 18, 2003, both of which are incorporated herein by reference.

### TECHNICAL FIELD

The disclosure pertains to methods and apparatus for visualization of microelectrode signals.

### BACKGROUND

Stereotactic surgical methods permit neurosurgeons to precisely target brain areas in the treatment of, for example, Parkinson's disease, seizure control, chronic pain, or other disorders. Typically microelectrodes are situated to detect electrical signals that are associated with local neuron activity at or near the microelectrodes. In some applications, such signals are processed to form so-called “spike trains” associated with a series of electrical spikes associated with neuron activity. Brain areas can be identified, targeted, or evaluated for treatment based on the time domain behavior of these microelectrode signals.

For example, in the treatment of Parkinson's disease, portions of the subthalamic nucleus (STN) can be targeted. Methods of selecting the targeted portion of the STN are non-standard among surgeons, and can be based on kinesthetic activity (response to movement), phasic activity (spike patterns), and tonic activity (firing rate). The analysis of phasic activity (spike patterns) depends largely on the surgeon's perception and interpretation of spike activity. Kinesthetic and tonic activity can be objectively evaluated based on characteristics of the spike train such as firing rate and interspike intervals, but such characteristics are highly variable and do not appear to be well suited for targeting. In addition, subjective factors such as selection of spikes from a spike train for inclusion in spike train analysis can contribute additional inconsistency. Additional clues such as the abrupt increase in background noise associated with the transition from the zona incerta (Zi) to the subthalamic nucleus (STN) due to the high density of cells in the STN region relative to the Zi can also be used.

While such microelectrode-based methods provide the surgeon with useful information, the existing methods are subjective and imprecise. Improved methods and apparatus for detection, characterization, and processing of microelectrode signals, and display of signals derived from microelectrode signals are needed.

### SUMMARY

Methods of visualizing neuronal signals include selecting at least one microelectrode electrical signal (MES) that is associated with a series of neuronal signals. The MES is processed to obtain an associated array of, and the array of values is displayed. In additional examples, the MES is processed to obtain a power spectral density or a probability density and the MES is classified based on the array of values. In additional examples, the MES is processed to form a spike train, and the array of values is associated with numbers of spikes in a first window and a second window, wherein the first window and the second window are adjacent windows and have predetermined durations. In further examples, the microelectrode signals are associated with a plurality of electrode insertion depths, and arrays of values associated with these depths are produced. In additional examples, the arrays of values are displayed as a function of insertion depth.

Apparatus according to the disclosure includes a sampler configured to receive a microelectrode electrical signal (MES) and produce a sampled representation of the MES. A memory is configured to store the sampled representation as a series of values, and a processor is configured to produce arrays of processed values based on the sampled representation and selected processing parameters. In additional representative examples, a processor input is configured to receive the selected processing parameters. In other examples, the processing parameters are associated with at least one of power spectral density and probability density. In additional examples, the processor input is configured to receive a window duration for at least a first window and a second window, and to produce the arrays of processed values based on numbers of spikes in the first window and the second window.

Display methods include receiving a plurality of microelectrode recordings associated with respective electrode insertion depths and producing an associated array of values for each recording. The associated array of values is displayed as a function of electrode insertion depth. In representative examples, the associated array of values is based on a power spectral density.

Methods of processing neuronal signals include receiving microelectrode recordings associated with respective insertion depths and estimating a rate of change of spike rate based on the received microelectrode recordings. In representative examples, the estimated rate of change of spike rate is displayed as a function of insertion depth and a brain feature is associated with an insertion depth based on the rate of change of spike rate. In representative examples, the rate of change of spike rate is estimated based on numbers of spikes in a first window and a second window.

A MER processing apparatus includes an input configured to receive a plurality of microelectrode recordings and a processor configured to produce an estimate of a rate of change of spike rate as a function of insertion depth based on the microelectrode recordings. In representative examples, a display is configured to display the rate of change of spike rate as a function of insertion depth and a classification engine is configured to produce a brain feature classifier based on the microelectrode recordings.

These and other features and advantages are described below with reference to the accompanying drawings.

### BRIEF DESCRIPTION OF THE DRAWINGS

FIG. 1 illustrates a trajectory of a deep brain stimulation (DBS) electrode.

FIG. 2 is a schematic diagram of a representative apparatus for acquisition, storage, and processing of microelectrode recordings.

FIG. 3 illustrates representative microelectrode recordings (MERS) obtained in different brain regions at different probe depths ranging from 23.1 mm to 34.1 mm.

FIG. 4A illustrates identifications of electrode depth with brain features during surgery based on MERs obtained from a Parkinson's disease patient. FIG. 4A is reproduced in black and white from a color original. Abbreviations are RT (reticular thalamus) shown in the color original in green, STN (subthalmic nucleus) shown in the color original in red, SNR (substania nigra reticula) shown in the color original in blue.

FIGS. 4B-4H contain representative color visualizations of processed MERs reproduced in black and white. In the color originals of FIGS. 4B-4H, amplitudes are color coded, wherein low amplitudes are shown in blue, high amplitudes are shown in dark red, and intermediate values are shown in intermediate colors. FIGS. 4C′-4E′ and 4C″-4E″ are alternative monochromatic representations of FIGS. 4C-4E, respectively.

FIG. 4B includes graphs of MER energy as a function of electrode depth for selected rank energies.

FIG. 4C represents MER power spectral density (PSD) as a function of electrode depth.

FIG. 4D represents MER marginal probability distribution function (mPDF) as a function of electrode depth.

FIG. 4E represents MER time series as a function of electrode depth.

FIGS. 4F-4H represent additional visualization angles for the representations of FIGS. 4C-4E, respectively.

FIGS. 4I-4J represent PSD and mPDF visualizations of MERs associated with 18 electrode trajectories, reproduced in black and white from color originals.

FIG. 4K includes visualizations of MERs obtained from a Parkinson's disease patient, reproduced in black and white from color originals.

FIG. 5A is a block diagram of method of processing spike trains.

FIG. 5B illustrates application of adjacent 8 bit windows to a portion of a binary representation of a spike train.

FIGS. 6A-6D are two dimensional histograms associated with spike counts in a first window and a second window for brain regions identified as GPI (globus pallidus internus), GPE (globus pallidus extremus), BRD (border cell), and TRM (tremor), respectively.

### DETAILED DESCRIPTION

Methods and apparatus are described that provide neurophysiological brainmaps of spontaneous neuronal discharges in the STN or other brain regions based on microelectrode recordings (MERs). Such methods and apparatus facilitate, for example, placement of deep brain stimulation (DBS) electrodes in the treatment of Parkinson's disease, and in the diagnosis, evaluation, and treatment of other diseases.

In typical DBS procedures, a probe is slowly inserted into a patient's brain in a stepwise manner. After each step, an electrical signal from the probe is recorded that is associated with neuron spiking at or near a probe tip. This electrical signal is referred to herein as a microelectrode electrical signal (MES), and can be processed into, for example, an audio signal, or displayed on an oscilloscope for use by a surgeon to confirm, identify, or characterize probe tip location. The probe path is typically precisely defined prior to surgery using, for example, magnetic resonance imaging (MRI), but during surgery, probe electrical signals are frequently the only direct indicator of probe placement. A stereotactic frame is generally used to position the probe, but MRI resolution and frame mechanical motion generally are such that it is difficult to precisely target regions such as the subthalmic nucleus (STN) or the globus pallidus internus (GPI). Neuronal activity differs in different regions, and can be used during surgery to confirm probe location. However, interpretation of neuron activity based on MERs is highly subjective, and MER processing to reduce such subjectivity can provide more reliable targeting.

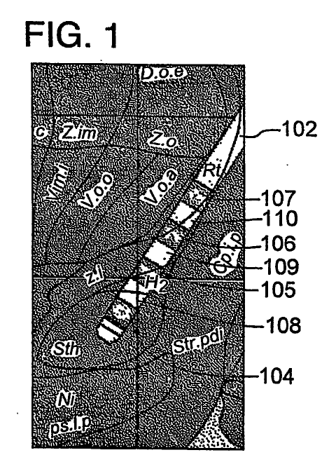

FIG. 1 illustrates an intended stereotactic trajectory for a DBS electrode 102 that includes stimulation surfaces 104, 105, 106, 107 separated by spacer surfaces 108, 109, 110. For the example of FIG. 1, the DBS electrode has a diameter of about 1.5 mm, and the stimulation surfaces 104, 105, 106, 107 are separated by about 1.5 mm. The DBS electrode 102 is shown with respect to several brain regions, including the subthalamic nucleus (Sth), the reticular thalamus (Rt), the zona incerta (Zi), and the substania nigra (Ni). Probe length, measured from probe tip to a ventral part of surface 107 is about 12 mm.

For some examples, spike trains are used that were obtained from eleven consecutive patients (8 males, 3 females) that underwent bilateral implantation of chronic deep brain stimulation in the subthalamic nucleus. Two patients who underwent general anesthesia during stereotactic surgery were omitted. Established surgical techniques were used. All of these recorded microelectrode trajectories were postoperatively analyzed. No patients received more than a single pass for any of the trajectories. In a representative example, MERs are recorded at each depth segment (each step) for about 30 seconds or longer. Some segments are recorded for shorter times because these segments are assumed to be prior to the thalamus based on probe depth and MER activity. The intended stereotactic trajectory is shown in FIG. 1.

A representative microelectrode recording (MR) apparatus 200 is illustrated in FIG. 2. A NELUROTREK electrode recording system 202, available from ALPHA OMEGA ENGINEERING, is in communication with a probe 204. The recording system 202 includes a sampler 206 configured to sample received neurophysiological signals at a selectable sample rate that can be, for example, between about 1000 Hz and 100 kHz. Typically, sampling rates of at least 5 kHz are selected. The recording system 202 also includes a hard disk 208 or other memory device configured to store the sampled data. The recording system 202 also includes a processor 212 configured to process the sampled data based on, for example, computer executable instructions provided by an input device such as a keyboard, or supplied via a network or a personal computer or otherwise provided. In an example, microelectrode signals can be produced with tungsten bipolar microelectrodes having 1000 Hz impedances between about 0.11Ω and about 0.43 MΩ. The recording system 202 can also include a spike discriminator that provides various spike discrimination analysis tools such as, for example, interspike interval (ISI) histograms and burst analysis. A display 210 and an audio output 214 such as a speaker permit visual and auditory analysis of MERs for distinguishing different structures along the electrode trajectory and identifying the target.

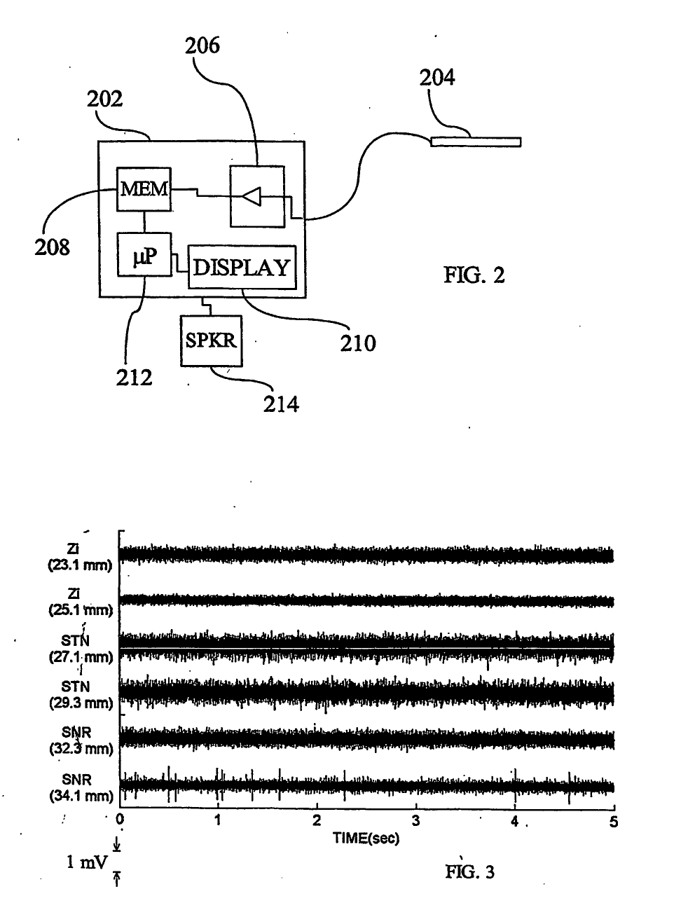

FIG. 3 displays microelectrode signals as a function of time for a selected Parkinson's disease patient at microelectrode depths between 23.1 mm and 34.1 mm along the stereotactic trajectory illustrated in FIG. 1. These signals are all associated with the patient's left hemisphere. Abbreviated annotations concerning location of the electrode with respect to particular features were provided during surgery, wherein the abbreviations used are: zona incerta (Zi), subthalamic nucleus (STN), and substania nigra reticulata (SNR).

For each electrode depth, portions of the recorded signal can be selected for analysis. For example, ten consecutive seconds that deviate the least from the mean can be selected. Segments shorter than 5 seconds can be omitted, and whole segments between 5-10 seconds long can be included. Segment energy can be calculated as the standard deviation of the signal amplitude. Rank energy can be evaluated by calculating the energy that is within the 25^{th}-75^{th} (P75), 10^{th}-90^{th} (P90), 5^{th}-95^{th} P95), and 1^{st}-99^{th} (P99) energy percentiles. Power spectral density can be calculated using, for example, Welch's method for nonparametric estimation of power spectral density (PSD), described in, P. D. Welch, “The Use of Fast Fourier Transform for the Estimation of Power Spectra: A Method Based on Time Averaging Over Short, Modified Periodograms,” IEEE Trans. Audio Electroacoust. AU-15:70-73 (1967). A marginal probability density function (mPDF) can be calculated to determine the distribution of the acquired signal with the signal mean subtracted. A time series of raw microelectrode signals can be obtained by low-pass filtering the signal with a low pass filter having a 4 Hz cutoff frequency. The resulting signal can be decimated to 200 samples, and the results plotted at the recorded electrode depth.

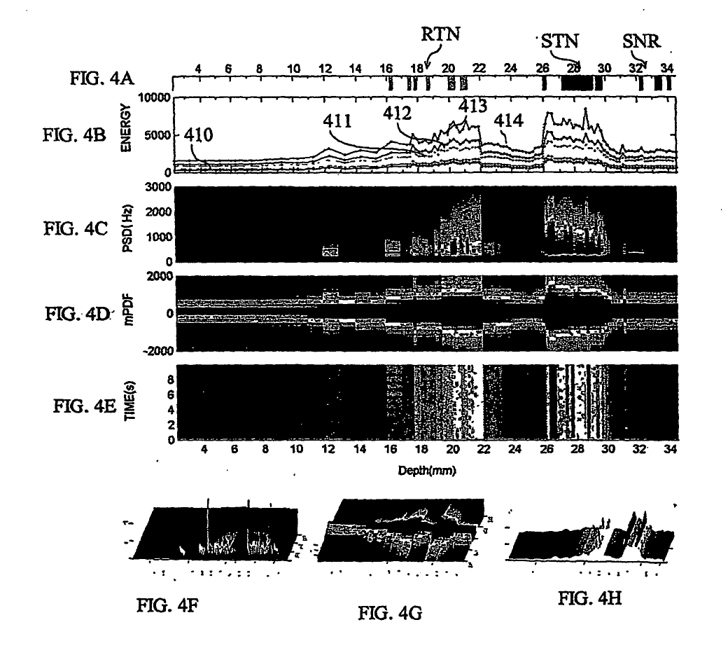

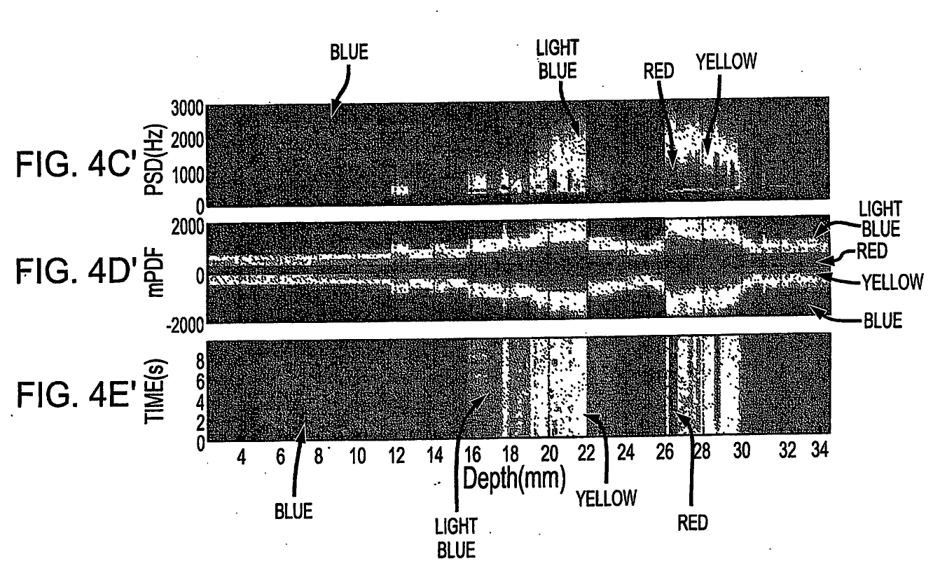

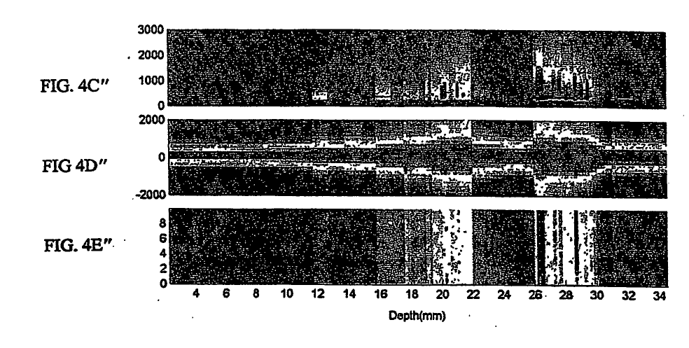

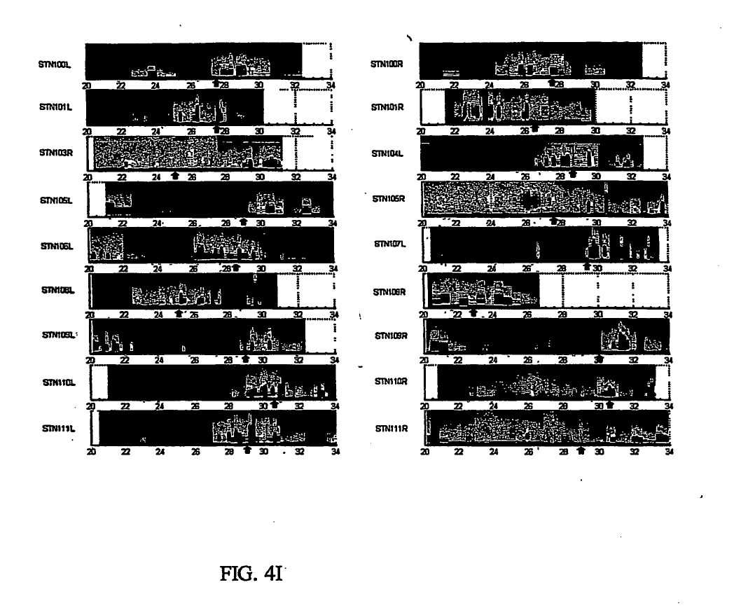

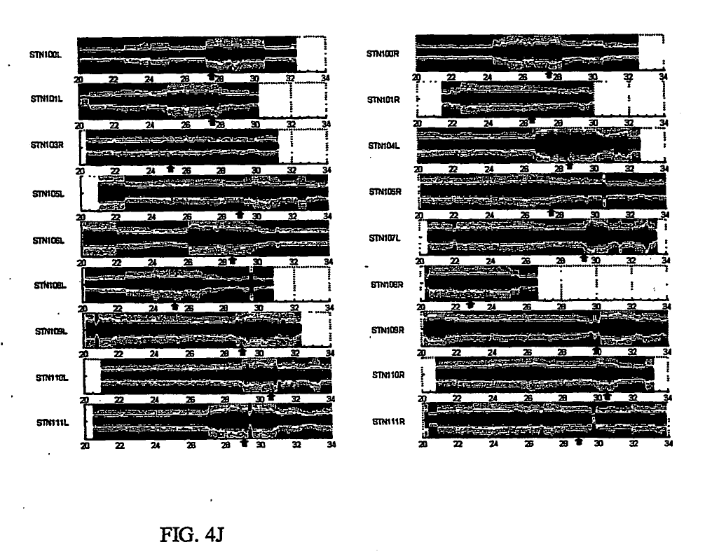

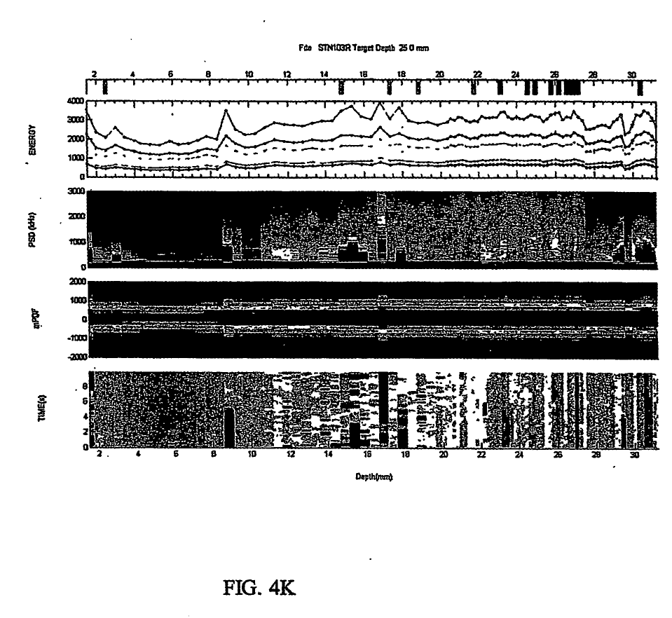

FIGS. 4A-4G include visualizations of statistical properties of MERs obtained from a Parkinson's disease patient Referring to FIG. 4A, selected depths were labeled as associated with brain regions RT, STN, and SNR, respectively, during surgery. FIG. 4B includes curves 410, 411, 412, 413, 414 associated with neuronal discharge energy, 25^{th}-75^{th} rank energy, 10^{th}-90^{th} rank energy, 5^{th}-95^{th} rank energy, and 1^{st}-99^{th} rank energy, respectively, Power spectral density (PSD) graphs, marginal probability density (mPDF) graphs, and time series graphs are shown in FIGS. 4C-4E, respectively, wherein low values are represented in blue and large values are represented in dark red, and intermediate values are represented using intermediate colors. The target structure is the subthalamic nucleus (STN) having a nominal target depth of 27.5 mm. These visualizations show boundaries of the target structure at depths of between 26 mm and 30 mm. FIGS. 4F-4H provide additional visualization angles for PSD, mPDF, and time series visualizations.

Referring to FIG. 4B, a distinct and abrupt increase in energy is associated with the STN. The different rank energies of FIG. 4B permit visual identification of potential outliers of the signal energy. For example, the P99 region demonstrates areas that show the largest outliers because it is associated with signal energies ranging from the 1^{st} to the 99^{th} percentile. As the signal energy range decreases, the mean signal energy is approached. The power spectral density (PSD) of FIG. 4C shows a distinct increase in power at higher frequencies in the region of the STN compared to the PSD at the Zona Incerta (Zi) and Fields of Forel (FF). A wider distribution of neuronal discharge amplitudes in the region of the STN in comparison to the Zona Incerta and the SNR is apparent in the mPDF plot of FIG. 4D. A ten-second time series of the microelectrode recording at each recorded depth as shown in FIG. 4E allows visualization of distinct neuronal firing patterns and amplitudes at different depths. While FIGS. 4A-4H all provide improved visualization, FIGS. 4C-4D (based on PSD and mPDF) are particularly convenient in distinguishing neuronal firing characteristics.

Some surgeries provide MER data for shorter or longer electrode trajectories, but the range of depths captured in the above figures includes the STN in all cases. A pre-surgery nominal target is typically about 27.5 mm for all patients, but the final target depth varies among patients, and between left and right hemisphere in the same patients. The final target depth for placement of the DBS is based on online auditory and visual analysis of raw MER signals and not on the visualization methods used to produce FIGS. 4A-4H.

Additional visualizations associated with 18 electrode trajectories are shown in FIGS. 4I-4J based on PSD and mPDF, respectively. The trajectories are identified with a six character patient-identifier (e.g., STN103) followed by “L” or “R” to indicate the associated hemisphere. Selected patient data is summarized in Table 1 and target depths and electrode impedances are summarized in Table 2.

TABLE 1
Selected patient information.

| Patient ID | Sex | Age | Disease duration (yrs) | Inclusion Criteria |
|---|---|---|---|---|
| STN 100 | F | 75 | 22 | IP, DID, OO |
| STN 101 | F | 57 | 21 | IP, DID, OO |
| STN 103 | F | 65 | 18 | IP, DID |
| STN 104 | M | 55 | 17 | IP, DID, OO |
| STN 105 | M | 75 | 20 | IP, BR, DID |
| STN 106 | M | 54 | 16 | IP, DID, OO, BR |
| STN 107 | M | 66 | 13 | IP, OO, DID |
| STN 108 | M | 61 | — | — |
| STN 109 | M | 66 | 20 | IP, OO, DID |
| STN 110 | M | 65 | 6 | IP, OO, DID, BR |
| STN 111 | M | 68 | 15 | IP, BR, DID |
| Average | | 63.9 | 15.6 | |

Abbreviations used are: idiopathic (IP), drug induced kinesia (DID), bradykinesia (BR), on/off fluctuations (OO), and tremor (TR).

TABLE 2
Target depths and electrode impedances.

| | |
|---|---|
| Final Target | Impedance |

| Patient ID | left | right | left | right |
|---|---|---|---|---|
| STN 100 | 27.5 | 27.5 | 0.21 | 0.21 |
| STN 101 | 27.5 | 26.5 | 0.3 | 0.3 |
| STN 103 | NA | 25 | NA | 0.36 |
| STN 104 | 28.6 | NA | 0.11 | NA |
| STN 105 | 29 | 27.5 | 0.25 | 0.2 |
| STN 106 | 28.5 | NA | 0.25 | NA |
| STN 107 | 29.3 | NA | 0.39 | NA |
| STN 108 | 25.1 | 22.8 | 0.4 | 0.45 |
| STN 109 | 29 | 30 | | 0.27 |
| STN 110 | 30.6 | 30.6 | | |
| STN 111 | 29 | 28.9 | 0.43 | |
| Average | 28.4 | 27.4 | 0.3 | 0.3 |

As the microelectrode is moved from the Zi to the STN as recorded in STN103R, 105R, STN110R, STN11R, it is apparent that low neuronal activity in the Zi is not necessarily followed by a large increase in PSD and/or mPDF. Differences in patient age, disease duration, disease inclusion criteria, and electrode impedance do not explain the lack of a signal transition from Zi to STN. However, these MERs are all associated with the right hemisphere, but patient handedness is unknown.

FIG. 4K contains visualizations of MERs obtained from a Parkinson's disease patient, and were obtained in a manner similar to that used to produce FIGS. 4A-4E. The Zi-STN transition is not readily apparent in the PSD or mPDF based visualizations. However, the time series visualization does permit brain structures along the stereotactic trajectory to be distinguished. Thus, multiple visualizations can be made available, and one or more of the visualizations selected for target identification or confirmation.

Substantial variations are apparent in visualization characteristics of the STN both among patients and in the left and the right hemispheres of the same patient. These differences may be associated with differences in degrees of neuronal degeneration in the STN or differences in the borders of degenerated regions. Such differences may also be associated with MER acquisition signal to noise ratios, variations in microelectrode location relative to the STN, and differences in impedance and/or microelectrode quality. As shown above, distinct regions of the microelectrode trajectories can be visualized even a variety of electrode impedances. The analysis and visualization methods shown above are robustness and simple, and can provide metrics for intra- and inter-clinical comparisons of target placements and the resulting clinical outcomes.

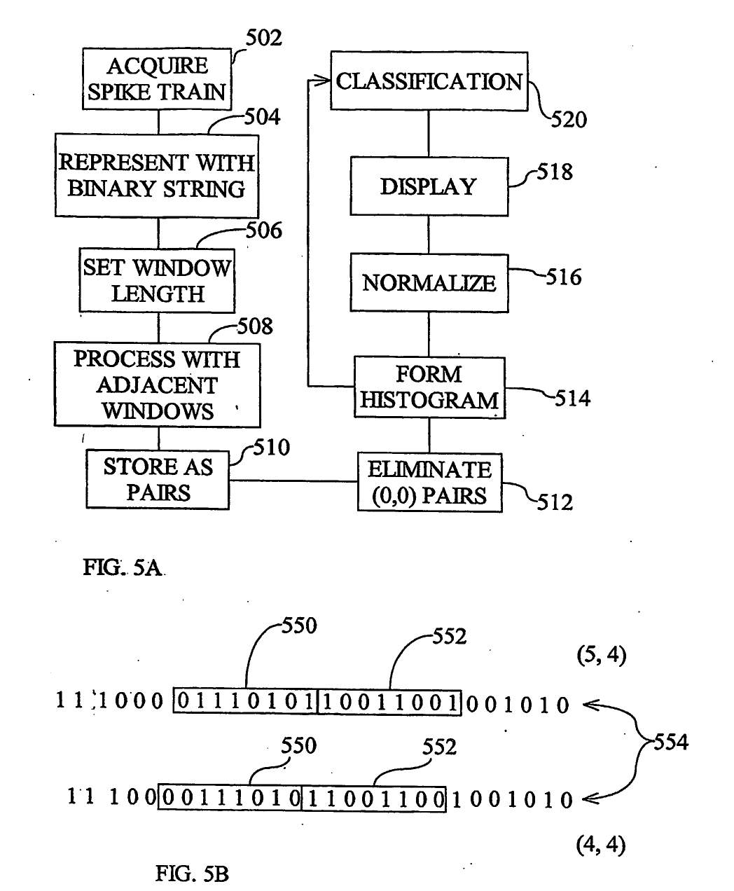

In another example of MER processing, analysis, and visualization, normal or diseased brain regions can be identified based on spike trains processed as illustrated in FIG. 5A. In a step 502, one or more spike trains is acquired, based on a series of spikes occurring in a time interval of between about 5 ms and 200 ms. In a step 504, a selected spike train is processed to produce a binary digital representation of the spike train in which the spike train is represented as a series of fixed duration intervals in association with a value of “0” or “1” that indicates whether or not a spike occurred in a particular interval. For example, a spike train having a duration of 5.7 sec can be represented as a series of 5700 1 ms intervals, and can be represented as an array that is 5700 units long. Each (binary) element of the array can be assigned a value associated with the presence or absence of a spike in the associated time interval. If a spike is detected at a tithe of, for example, 0.1189 s from the beginning of the spike train, a value of “1” indicating that a spike occurred can be associated with an interval value 119. Schematically, such a representation of a spike trains can be written as a series binary digits 0, 0, . . . , 1, . . . , 0 or as a two dimensional array, or otherwise represented. In this way, a digitized spike train (DST) is produced that is a series of binary values. Generally several or many of the time intervals are associated with spikes, but only a spike at a single interval is indicated in this example.

In a step 506, window durations for a first window and a second window are selected, and in a step 508, the DST is processed based on a number of “1”s in windows of the first duration and the second duration. Typically, the first and second widows are adjacent and have the same window duration, but non-adjacent windows and windows of different durations can be used. Window duration can be expressed in terms of window length in bits based on a sampling rate used to obtain the spike trains.

In an example, a single window length of eight bits is selected, and 8-bit words based on binary digits within each window are formed for all, or substantially all binary values in the DST. For example, using adjacent 8-bit windows on a binary digit series 0111010110011001 a value of 5 is associated with a first window (first 8 bits) and a value 4 associated with a second window (second 8 bits). FIG. 5B illustrates a first window 550 and a second window 552 situated with respect to a DST such to obtain integer pairs (5, 4) and (4, 4). The first are second windows are moved as so-called “sliding” windows through the DST to produce a series of such integers pairs. These pairs are stored in a step 510.

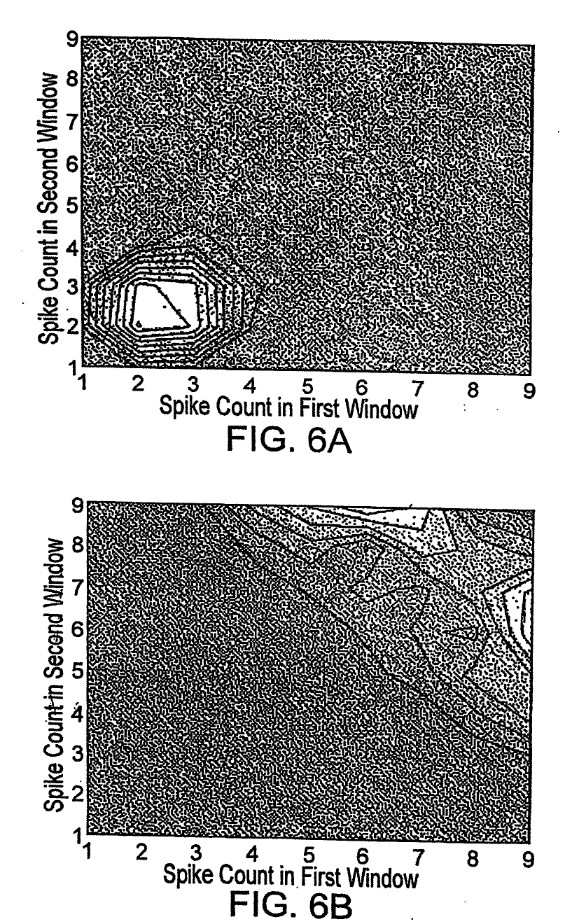

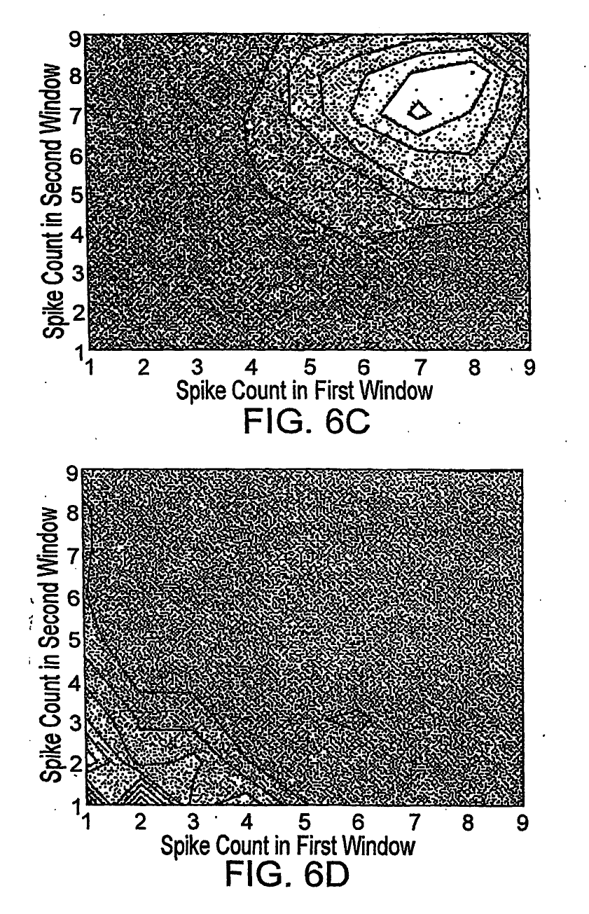

In a step 512, the integer pair (0, 0) is removed and the remaining integer pairs are binned together to create a two dimensional histogram in step 514. Such histograms can be normalized by dividing by a total number of entries in a step 516, and histogram values converted to associated natural logarithms. Normalization is particularly suited for applications in which spike trans of different lengths are processed, as differences attributable to spike train length are reduced. Histograms are displayed in a step 518. Representative histograms generated with a 100 Hz sampling rate and a window size of 9 bits are shown in FIGS. 6A-6D for cells of type GPI, GPE, BRD, and TRM respectively. Count densities are represented using different gray values.

A one dimensional histogram, based on a single moving window, is associated with a distribution of spike rates. The two dimensional histogram can be associated with changes in spike rates. For example, a particular histogram based on GPE spike trains sampled at 1000 Hz for the DST and with a 20 bit window size can have relatively large values associated with the (4, 18) and the (10, 10) bins. These values indicate that if four spikes occur in a 20 ms period, it is likely that there will be 18 spikes in a next 20 ms period. Similarly, if 10 spikes occur in a particular window, it is relatively likely that 10 spikes will occur in the next window. Dual window processing is convenient, but other processing methods associated with a rate of change of spike rate can be used.

Display of dual window spike train histograms permits identification of a particular brain feature. As is apparent from FIGS. 6A-6D, histograms associated with different brain regions occupy different areas on a two dimensional histogram graph. Thus, classification methods such as, for example, support vector machines, can associate a MER with a particular brain region. Such methods can provide an estimate of a boundary between the histogram graph areas that can be used to assign a particular signal to a particular brain region. Thus, an additional classification processor can be used to distinguish various brain features based on processed spike trains in a step 520.

Support vector machines (SVMs) or other classifiers can be used to distinguish and provide boundaries, for example, between GPI, GPE, BRD, and TRM cells. Such support vector machines can be conveniently implemented using support vector libraries available for MATLAB technical computing software available from The Math Works. In an example, two data sets were processed using a dual window technique. A first data set, referred to as a “dirty” data set (DDS), included 93 spike trains. While the DDS was collected under normal surgical conditions, expert labels applied to these spike trains were supplied outside of surgery. The DDS was randomly divided into a test data set and a training subset. The training subset was used to classification algorithm development, and the test subset was used for validation. The second data set, referred to as a “clean” data set (CDS) included 47 spike trains recorded for training neurosurgeons in MER signal evaluation.

Support vector machines (SVMs) were developed based on these data sets, and leave-one-out cross validation used during algorithm development to test algorithm feature extraction effectiveness. Tables 3-4 below contain confusion matrices associated with cross validation using the CDS and the training set of the DDS, respectively. Upon completion of algorithm development, the algorithm was applied to the test subset of the DDS. Table 5 shows the confusion matrix associated with the algorithm based on the training subset.

TABLE 3
Confusion Matrix for Leave-One-Out Cross Validation of CDS

SVM

| EXPERT | GPE | GPI | BRD | TRM |
|---|---|---|---|---|
| GPE | 13 | | | |
| GPI | | 9 | | |
| BRD | | | 7 | |
| TRM | | | | 8 |

TABLE 4
Confusion Matrix for Leave-One-Out Cross Validation
of the Training Set of the DDS

SVM

| EXPERT | GPE | GPI | BRD | TRM |
|---|---|---|---|---|
| GPE | 31 | | 2 | |
| GPI | 1 | 2 | | 1 |
| BRD | 2 | | 5 | |
| TRM | 2 | | | 1 |

TABLE 5
Confusion Matrix for DDS Test Subset Using
Training Subset Based SVM.

SVM

| EXPERT | GPE | GPI | BRD | TRM |
|---|---|---|---|---|
| GPE | 30 | | 2 | |
| GPI | 2 | 6 | | |
| BRD | | | 3 | |
| TRM | 3 | | | |

As shown in the above tables, the SVM classifier for the CDS identified neuron types with perfect accuracy. SVM classifiers associated with the DDS were less reliable, but still provide reasonable accuracy even in the presence of noise and or signal artifacts.

The visualization methods and apparatus described above facilitate electrode placement, permit objective comparisons regarding electrode placement, trajectory accuracy, and treatment outcomes. In addition, these methods permit display of the full time evolution of MER signals so that a surgeon need not rely solely on memory of an acoustic signal or oscilloscope trace to evaluate MER signal time evolution.

Representative methods and apparatus have been described. It will be apparent that these methods and apparatus can be modified in arrangement and details. Method steps can be carried out in different orders, and one or more steps can be omitted. The methods can be implemented based on computer executable instructions stored in a computer readable medium such as a hard disk or other disk, or memory. Visualization and classification can be performed in diagnosis, treatment, or evaluation, before, during, or after surgery. In addition, other types of electrical, audio, or other signals can be similarly processed. The representative examples described are not to be taken as limiting, and we claim all that is encompassed by the appended claims.

We claim:

1. A method, comprising:
selecting at least one microelectrode recording (MER);
processing the at least one MER to obtain an associated array of values; and
displaying the array of values.

2. The method of claim 1, wherein the MER is processed to obtain a power spectral density or a probability density.

3. The method of claim 1, wherein the at least MER is selected based on an insertion depth at which the at least MER is recorded.

4. The method of claim 1, further comprising classifying the at least one MER based on the array of values.

5. The method of claim 1, further comprising processing the MER so that the array of values is associated with numbers of spikes in a first window and a second window.

6. The method of claim 5, wherein the first window and the second window are adjacent windows and have predetermined durations

7. The method of claim 5, wherein the first window and the second window are adjacent windows having a common duration.

8. The method of claim 1, wherein MERs associated with a plurality of electrode insertion depths are selected, and corresponding arrays of values are produced.

9. The method of claim 8, wherein the arrays of values are displayed as a function of insertion depth.

10. An apparatus, comprising:
a sampler configured to receive a microelectrode electrical signal (MES) and produce a sampled representation of the MES;
a memory configured to store a series of values based on the sampled representation; and
a processor configured to produce arrays of processed values based on the sampled representation and selected processing parameters.

11. The apparatus of claim 10, further comprising a processor input configured to receive the selected processing parameters.

12. The apparatus of claim 10, wherein the processing parameters are associated with at least one of power spectral density and probability density.

13. The apparatus of claim 10, wherein the processor input is configured to receive a window duration for at least a first window and a second window, and the processor is configured to produce the arrays of processed values based on numbers of spikes in the first window and the second window.

14. A display method, comprising:
receiving a plurality of microelectrode recordings associated with respective electrode insertion depths;
producing an associated array of values for each recording; and
displaying the associated array of values as a function of electrode insertion depth.

15. The method of claim 14, wherein the associated array of values is based on a power spectral density.

16. A method, comprising:
receiving microelectrode recordings associated with respective insertion depths; and
estimating a rate of change of spike rate based on the received microelectrode recordings.

17. The method of claim 16, further comprising displaying the estimated rate of change of spike rate as a function of insertion depth.

18. The method of claim 16, further comprising associating a brain feature with an insertion depth based on the rate of change of spike rate.

19. The method of claim 16, wherein the rate of change of spike rate is estimated based on numbers of spikes in a first window and a second window.

20. An apparatus, comprising:
an input configured to receive a plurality of microelectrode recordings;
a processor configured to produce an estimate of a rate of change of spike rate as a function of insertion depth based on the microelectrode recordings.

21. The apparatus of claim 20, further comprising a display configured to display the rate of change of spike rate as a function of insertion depth.

22. The apparatus of claim 20, further comprising a classification engine configured to produce a brain feature classifier based on the microelectrode recordings.

23. A processing method, comprising:
receiving a microelectrode recording;
processing the microelectrode recording to produce an array of processed values; and
associating the microelectrode recording with a particular brain region based on the processed values.

24. The method of claim 23, wherein the processed values are associated with a power spectral density.

25. The method of claim 23, wherein the processed values are associated with a rate of change of spike rate.
